(19) United States
(12) Patent Application Publication
(10) Pub. No.: US 2012/0109184 A1
(43) Pub. Date: May 3, 2012

(54) VIBRATORY MOTOR USE

(75) Inventors: James T. Spivey, Cincinnati, OH (US); Craig N. Faller, Milford, OH (US)

(73) Assignee: ETHICON ENDO-SURGERY, INC., Cincinnati, OH (US)

(21) Appl. No.: 12/916,774

(22) Filed: Nov. 1, 2010

(51) Int. Cl. A61B 17/28; A61H 1/00

(52) U.S. Cl. 606/205; 601/46

(57) ABSTRACT

Devices and methods are provided for reducing the effect of frictional forces on the operation of a surgical instrument, particularly when inserted within a tortuous body lumen. A vibratory mechanism coupled to the surgical instrument can be activated to vibrate the shaft and/or the actuator of the surgical instrument to reduce the effect of frictional forces generated between the shaft and the actuator of the surgical instrument, thereby reducing the force necessary to actuate the end effector.

### FIELD OF THE INVENTION

The present invention relates to devices and methods for performing surgical procedures, and more particularly to surgical instruments having a vibratory mechanism coupled thereto and methods of operating the same.

### BACKGROUND OF THE INVENTION

Minimally invasive surgical procedures allow surgeons to utilize natural body orifices or small incisions in the body wall to deliver instrumentation to an internal surgical site. Minimally invasive techniques are becoming increasingly common due to the benefits associated with the techniques' focus on minimizing surgical trauma. Such procedures advantageously reduce post-operative pain and recovery time, decrease the possibility of wound infection, and improve cosmetic outcomes as compared to conventional open medical procedures.

Many minimally invasive procedures are performed through an endoscope (including without limitation the use of laparoscopes and surgical trocars or percutaneous applications). Suitable surgical instruments (e.g., graspers, dissectors, scissors, retractors, etc.) can then be inserted through the endoscope such that the surgeon can position, manipulate, and view the instruments and associated accessories inside the patient through a relatively small access opening. Laparoscopy is the term used to describe an “endosurgical” approach in which surgical instruments are delivered to an internal surgical site generally through trocars placed in a surgical incision made through the body wall. Another minimally invasive surgical technique is the percutaneous approach. With percutaneous surgery, a small diameter device is placed directly through the body wall to the surgical site. The entry site requires little or no closure (frequently a band-aid will suffice). Still less invasive treatments include those that are performed endoscopically through a natural body orifice. Examples of this approach include, but are not limited to cystoscopy, hysteroscopy, esophagogastroduodenoscopy, and colonoscopy.

The rise in popularity of minimally invasive surgeries and surgeons' increasing comfort with endoscopic techniques has led to significant development with respect to endoscopic procedures and the instrumentation that can be used in such procedures. Recently, many surgical devices have been made that can be inserted through the working channel of an endoscope. For example, some endoscopic surgical devices include a flexible tubular shaft, a control member longitudinally and/or rotationally movable relative to the tubular shaft, an end effector coupled to the distal ends of the tubular member and the control member, and a housing with controls for actuating the control member. Actuation of the control member relative to the tubular shaft can cause operation of the end effector. Accordingly, the ability to cut or grasp tissue, apply fasteners, or perform various other procedures through an endoscope permits myriad minimally invasive surgical solutions to medical problems, especially those of the gastrointestinal tract. Alternatively, for laparoscopy or percutaneous approaches devices may have rigid tubular shafts with articulating end effectors, i.e., that can be angularly oriented relative to the shaft.

However, working space is limited in minimally invasive procedures due to the reduced size of the access opening (natural or otherwise) and the frequent need to concurrently deliver multiple instruments to the surgical site. Accordingly, one drawback of current devices results from frictional forces generated between tightly-compacted components of the surgical instruments delivered through narrow channels (particularly devices that are flexible or articulating). This is especially true in procedures employing flexible endoscopes in which flexible surgical instruments are inserted along a tortuous path (e.g., endoscopically through a tortuous body lumen). As a flexible surgical instrument is inserted through the mouth and esophagus, for example, frictional forces can be amplified at locations at which the surgical device bends, curves, or articulates, thereby impairing the performance of the device by interfering with the ability of the components to move relative to each other.

Accordingly, there remains a need for improved methods and devices that reduce the effect of frictional forces on the operation of endoscopic surgical instruments.

### SUMMARY

The present invention generally provides surgical devices and methods that include the use of a vibratory mechanism to reduce the effect of frictional forces on the operation of the device. In one embodiment, a surgical instrument is provided having a flexible elongate shaft, an end effector, and a flexible elongate actuator for actuating the end effector. The flexible elongate shaft has a proximal end coupled to a handle and a distal end for insertion into a patient. The actuator extends through the shaft between the handle and the end effector. A vibratory mechanism is coupled to the instrument for vibrating the shaft and/or the actuator to reduce the frictional forces (static and dynamic) generated between the shaft and the actuator when the shaft is inserted into a patient, thereby reducing the force required to actuate the end effector.

The shaft can be inserted into a patient, for example, through a tortuous body lumen. In one embodiment, the shaft extends distally from a handle that can be disposed outside a patient. The handle can include, for example, a dampener to reduce vibrations transmitted to the handle.

The end effector can effect tissue and can be coupled to or disposed on the distal end of the shaft. In one embodiment, the end effector includes opposed jaws that can be moved from an open position to a closed position.

The actuator extends through the shaft between the handle and the end effector and is effective to actuate the end effector. In one embodiment, the actuator can be a flexible, elongate cable, wire, or wire coil. To actuate the end effector, the actuator can move relative to the shaft. For example, in one embodiment, the actuator can rotate relative to the shaft such that the actuator applies a rotational force to the end effector to rotate the end effector. In another embodiment, the actuator can translate longitudinally relative to the shaft such that the end effector is actuated, for example, to move opposed jaws of the end effector between open and closed positions.

The vibratory mechanism can also have various configurations and it can be disposed at various locations on or in the device. In one embodiment, the vibratory mechanism is disposed within the elongate shaft for vibrating the shaft and/or the actuator to reduce the frictional forces between the shaft and the actuator. By way of example, the vibratory mechanism can be a mechanical vibrator, which can be manually actuated, a vibratory motor, an ultrasonic transducer, or other commonly known mechanisms to introduce an oscillation. The vibratory mechanism can be fixedly coupled to the device, or it can be removably and/or replaceably coupled to the device.

In other aspects, a surgical device is provided having a handle, a flexible elongate shaft extending distally from the handle, an end effector for effecting tissue disposed on a distal end of the elongate shaft, and a flexible elongate actuator that extends through the shaft and is movable relative to the shaft to effect movement of the end effector. The surgical device also includes a vibratory mechanism associated with the shaft and/or actuator for converting a static frictional force between the shaft and the actuator when the shaft is disposed in a tortuous body lumen to a dynamic frictional force to reduce the force necessary to actuate the end effector. The device can have various configurations as described above, and as set forth in more detail herein.

In another embodiment, a surgical method is provided that includes advancing the flexible elongate shaft of a surgical instrument through a tortuous body lumen. The distal end of the flexible elongate shaft includes an end effector that is positioned within the body. A flexible elongate actuator extending through the elongate shaft can be actuated to actuate the end effector. A vibratory mechanism coupled to the surgical instrument can vibrate the shaft and/or the actuator to reduce a frictional force generated between the shaft and the actuator when the shaft is positioned within the tortuous body lumen, thereby reducing the force necessary to actuate the end effector.

In one embodiment, the flexible elongate actuator can translate longitudinally through the elongate shaft upon actuation. Alternatively or in addition, the flexible elongate actuator can rotate during actuation to rotate the end effector. For example, in one embodiment, actuating the actuator can open and close opposed jaws of the end effector.

In another embodiment, the vibratory mechanism can be activated prior to actuating the flexible elongate actuator. Alternatively or in addition to, the vibratory mechanism can be activated concurrently with the actuation of the actuator.

### BRIEF DESCRIPTION OF THE DRAWINGS

The invention will be more fully understood from the following detailed description taken in conjunction with the accompanying drawings, in which:

FIG. 1 illustrates one exemplary embodiment of a surgical device having a vibratory mechanism coupled thereto, the device being disposed through an endoscope inserted into the upper gastrointestinal tract of a patient;

FIG. 2 is a side view of the surgical device of FIG. 1;

FIG. 3 is a partially cross-sectional view of the handle of the surgical device of FIG. 1;

FIG. 4A is a side view of one embodiment of an end effector for use with the surgical device of FIG. 1, the end effector having opposed jaws and being in an open position;

FIG. 4B is a side view of the end effector of 4A showing the opposed jaws in a closed position;

FIG. 4C is another side view of the end effector of FIG. 4A, with the end effector rotated following rotational actuation.

FIG. 5 is a partially cross-sectional view of another embodiment of a handle of a surgical device having a vibratory mechanism coupled thereto;

FIG. 6A is a side view of another embodiment of a surgical device having a vibratory mechanism coupled thereto;

FIG. 6B is a view of the vibratory mechanism coupled to the surgical device of FIG. 6A.

FIG. 7 is a side view of another exemplary embodiment of a surgical device having a vibratory mechanism coupled thereto; and

FIG. 8 is a partially cross-sectional view of the handle of the surgical device of FIG. 7.

### DETAILED DESCRIPTION

Certain exemplary embodiments will now be described to provide an overall understanding of the principles of the methods and devices disclosed herein. One or more examples of these embodiments are illustrated in the accompanying drawings. Those skilled in the art will understand that the methods and devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides surgical methods and devices in which a vibratory mechanism can be used to reduce the effect of frictional forces on the operation of the device, and in particular when the device is inserted into a patient through a tortuous body lumen. For example, when a flexible shaft having an actuator disposed therethrough is inserted through a tortuous body lumen, frictional forces between the shaft and the actuator can interfere with the actuation of an end effector operationally coupled to the actuator and disposed on the distal end of the elongate shaft. The use of a vibratory mechanism coupled to the surgical device can be effective to reduce any frictional force between the shaft and the actuator that interferes with their relative movement, thereby reducing the force necessary to actuate the actuator. For example, the vibratory mechanism can be effective to convert a static frictional force between the shaft and the actuator to a dynamic frictional force.

The devices or instruments for use with the vibratory mechanisms described herein can have a number of different configurations. Although in certain exemplary embodiments, the surgical device or instrument generally includes a shaft for insertion into a patient, an end effector for effecting tissue, and an actuator extending through the shaft for effecting operation or movement of the end effector, a person skilled in the art will appreciate that the concepts described herein can be applied to other surgical, therapeutic, or diagnostic devices in which it is desirable to reduce the effect of frictional forces on the operation of a device.

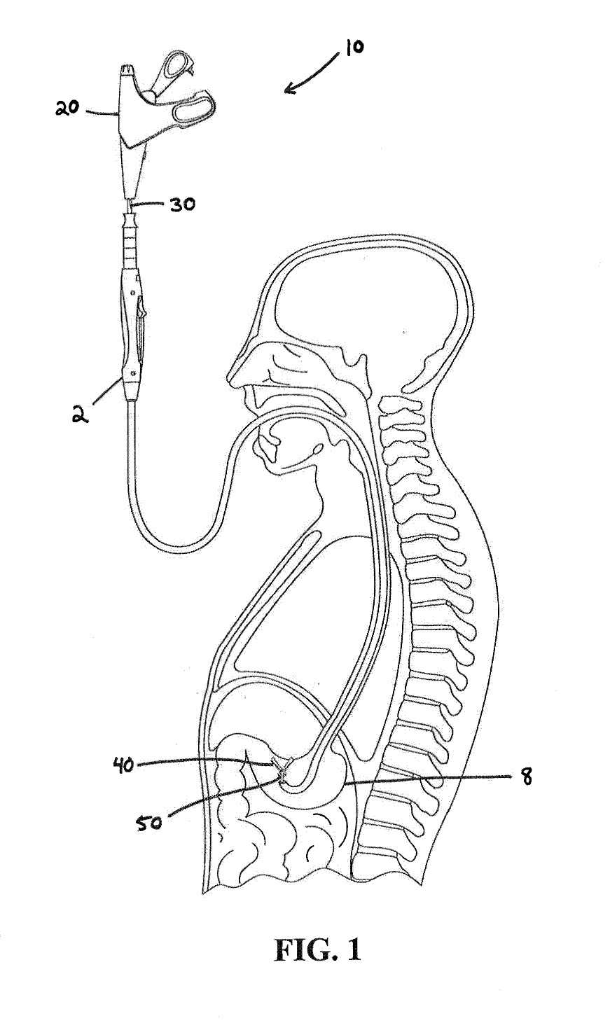

FIGS. 1-4C depict one exemplary embodiment of a surgical device 10 having a vibratory mechanism 60 coupled thereto. As illustrated in FIG. 1, the surgical device 10 can be disposed within a tortuous lumen to position the end effector 40 at an internal surgical site. As shown, the surgical device 10 is disposed within an endoscope 2 for transesophageal access to a surgical site within the stomach 8 of a patient. Although the illustrated surgical device 10 is delivered to the surgical site through the mouth and esophagus, it will be appreciated by a person skilled in the art that the surgical device 10 can be delivered to an internal surgical site through any opening within a patient's body, whether a natural orifice (e.g., anally or vaginally) or a surgical opening (e.g., through a trocar in a percutaneous incision or a purely percutaneous approach). The surgical device 10 depicted in FIG. 1 can also be delivered directly to the surgical site through the orifice or opening directly, without the use of an auxiliary endoscope, trocar, or other access port.

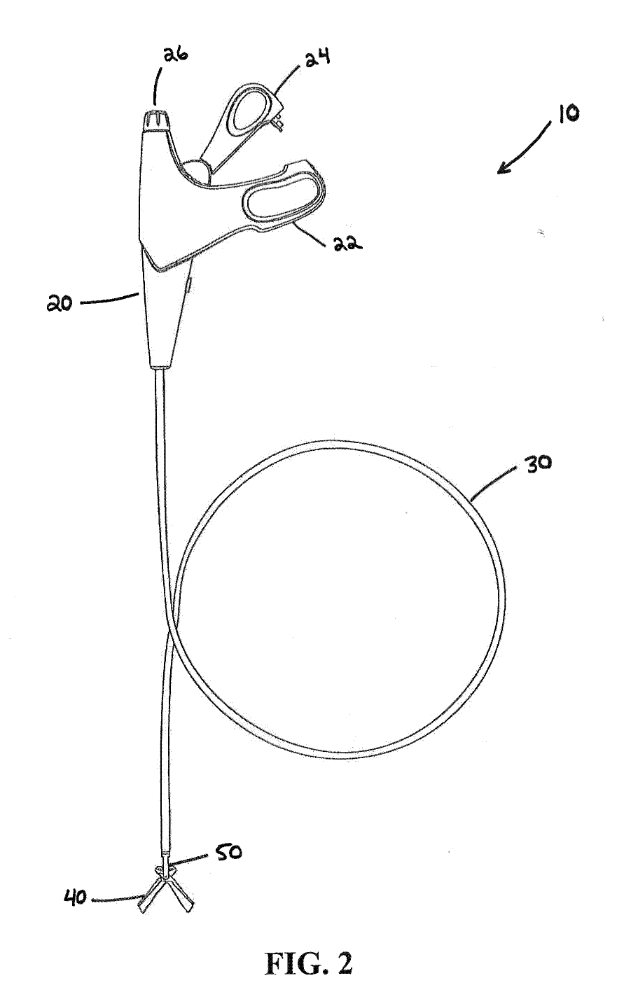

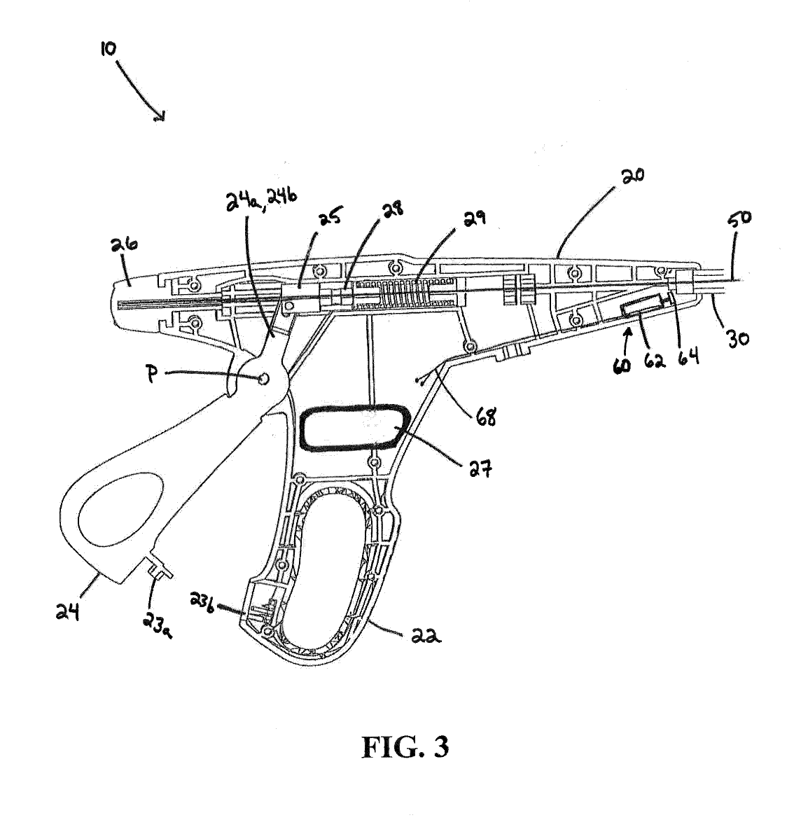

As shown in FIGS. 2 and 3, the surgical device 10 generally includes a handle assembly (or handle) 20, an elongate shaft 30 extending distally from the handle 20, an end effector 40 for effecting tissue disposed on a distal end of the elongate shaft 30, and an actuator 50 extending between the handle 20 and the end effector 40. The surgical device 10 can also include a vibratory mechanism 60 coupled thereto to vibrate at least one of the shaft 30 and the actuator 50 to reduce the frictional force generated between the shaft 30 and the actuator 50, as will be described in detail below.

The handle 20 can have a variety of configurations, but is generally configured to be positioned outside the patient's body to facilitate control of the device 10. A person skilled in the skill in the art will appreciate that any of the various handle assemblies known in the art can be used including, for example, scissor-grip, pistol-grip, spool style handles, syringe style handles, and various other handle configurations modified in accordance with the teachings herein.

In the embodiment illustrated in FIG. 2, the handle 20 is generally pistol-shaped and includes a stationary grip 22 extending from a bottom surface of the handle 20. The handle 20 also includes a trigger 24 and knob 26, both of which are moveably coupled to the handle 20. As will be discussed in detail below, the trigger 24 is pivotally coupled to the handle and can be effective to move the actuator 50 longitudinally relative to the shaft 30 to actuate the end effector 40. The rotatable knob 26 is rotatably coupled to the handle 20 and can be effective to rotationally move the actuator 50 relative to the shaft 30 to rotate the end effector 40, as will also be discussed in detail below. Alternatively, the rotatable knob 26 may also be connected to the shaft 30 allowing the entire shaft 30/end effector 40 to rotate relative to the handle 20. While not shown, the handle 20 can include additional or alternative actuators configured to effect various motions, such as articulation of the end effector, firing, etc. The illustrated trigger 24 and knob 26 can also be used to effect other motions as well.

Referring now to FIG. 3, the trigger 24 of the handle 20 can pivot about a pivot point (P) in a direction towards and away from the stationary grip 22. The illustrated trigger 24 has a pair of arms 24a, 24b that couple the trigger 24 to a shuttle assembly 25. The actuator 50 is received at the distal portion of the handle 20 and extends proximally to couple to the shuttle assembly 25. Within the shuttle assembly 25, the actuator 50 can extend proximally through a collar 28 to couple to the rotation knob 26. The actuator 50 can be securely fastened to the collar 28 such that the actuator 50 cannot translate distally and proximally with respect to the collar 28.

In use, the operator can place one or more fingers through the grip 22, for example, and manipulate the trigger 24 with a thumb to move the trigger 24 towards the grip 22. Accordingly, the arms 24a, 24b of the trigger 24 pull the shuttle assembly 25 proximally, thereby pulling the collar 28 and actuator 50 proximally. A spring 29 can bias the shuttle assembly 25 in its distal position such that when the trigger 24 is released, the spring 29 can move the shuttle assembly 25, collar 28, and actuator 50 distally. Alternatively, the spring 29 can be integrated in series between the shuttle 25 and the actuator 50 such that the spring 29 acts as a force limiter thereby reducing the effective force experience in the shaft and end effector components resulting in a more robust/reliable system. Another possible outcome of a series spring configuration is to limit the force experience at the end effector to improve clinical outcomes or, in the case of surgical staplers or clip appliers, provide more consistent forming loads. As shown, the trigger 24 can also include a lock element 23a that can be securely received within a lock cavity 23b formed in the grip 22 to allow the operator to secure the trigger 24, and thus the shuttle assembly 25, actuator 50, and end effector 40, in a given position. Various other locking mechanisms known in the art can also or alternatively be used, including pawl and ratchet mechanisms, gears, etc.

As discussed above, the handle 20 can additionally or alternatively include a rotatable knob 26 for rotating the actuator 50 relative to the shaft 30. The rotatable knob 26 can include a lumen or bore formed therein that receives the proximal end of the actuator 50. The lumen is shaped to allow free slidable movement of the actuator 50 along its axis, and to rotationally couple the proximal end of the actuator 50 to the knob 26. As a result, when the knob 26 is rotated to rotate the actuator 50, a torque is generated which causes rotation of the actuator 50 within the shaft 30, and ultimately rotation of the end effector 40 to which the actuator 50 can be coupled. Other rotation mechanisms known in the art can also or alternatively be used, and the rotation mechanism can be disposed anywhere on the handle and/or the elongate shaft for rotating the end effector relative to the shaft and/or for rotating the entire shaft w/ the end effector coupled thereto relative to the handle.

The elongate shaft 30 can also have a variety of configurations, but generally extends distally from the housing and defines a lumen through which the actuator 50 can extend. A person skilled in the skill in the art will appreciate that any of the various shafts for endoscopic, laparoscopic, percutaneous, and other minimally invasive surgical devices known in the art can be modified in accordance with the teachings herein.

In an exemplary embodiment, at least a portion of the elongate shaft 30 is flexible or semi-flexible to allow the shaft 30 to be inserted into a patient translumenally, e.g., through a natural orifice, an endoscope, or a surgical incision. While various materials and techniques can be used to form the shaft, the elongate shaft 30 can be formed from a friction reducing flexible outer sheath having a flat coil wire extending therethrough. The flexibility of the shaft 30 can vary along its length and the shaft 30 can be formed from one or more components that are mated together.

As shown in FIG. 3, the proximal end of the shaft 30 is received within the distal portion of the handle 20. The shaft 30 can be integral with the handle 20 or it can be fixedly or removably coupled to the handle 20. Additionally, the shaft 30 can be configured to move (e.g., rotate or move longitudinally) relative to the handle 20. As will be discussed in detail below, the shaft 30 can be in contact with the vibratory mechanism 60 such that the shaft 30 vibrates in response to activation of the vibratory mechanism 60.

The actuator 50 can also have a variety of configurations, but generally extends distally from the handle 20 to the end effector 40 in order to actuate the end effector 40. One skilled in the art will appreciate that the actuator can have any number of configurations, shapes, and sizes depending at least in part on the configuration of the shaft, the end effector to which it will be coupled, and the motion to be effected. For instance, the actuator 50 can extend through the elongate shaft 30, or it can be disposed around the elongate shaft, with the elongate shaft forming an internal stationary member that traverses the interior lumen of the actuator and that connects terminally to the end effector 40. The proximal end of the stationary inner elongate shaft may be fixedly connected to the handle 20. Movement of the trigger 24 can impart motion to the actuator 50 resulting in actuation of the end effector 40.

The actuator can be made from any suitable material, for example, a cable or metal wire (e.g., a multi-layered steel cable, such as a tri-layered steel cable). The actuator is preferably formed from a flexible or semi-flexible material, such as a nickel-titanium alloy or stainless steel, which permits the actuator to transmit torque by rotation without taking a cast, and with minimal whipping. The actuator also preferably has a sufficiently large diameter to sufficiently transmit longitudinal force and torque to the distal end of the actuator, yet not so large that the actuator is prevented from flexing as the elongate shaft is passed through a tortuous lumen.

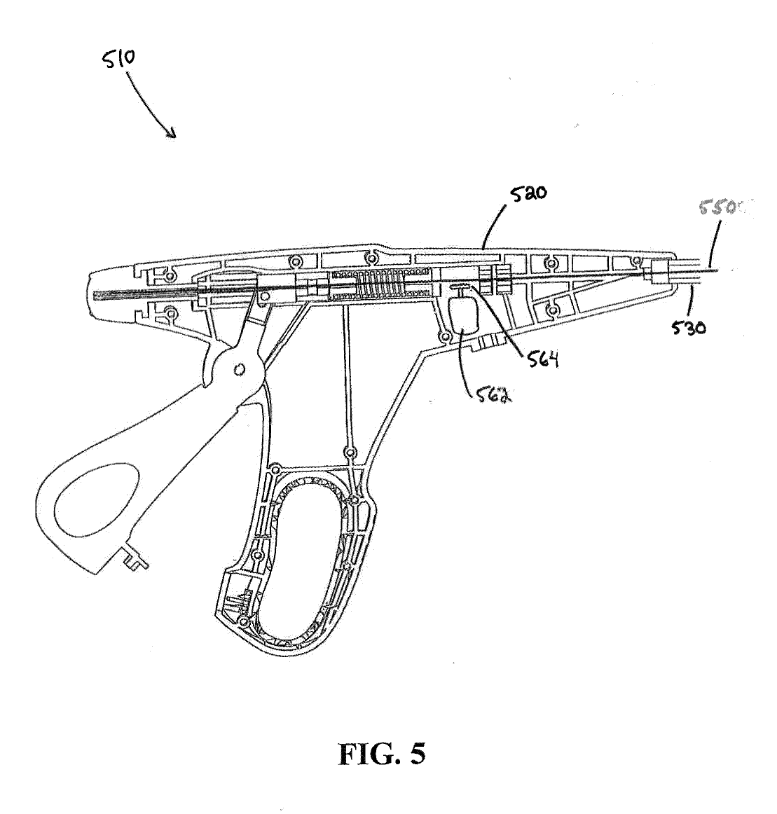

As will be discussed in detail below in reference to the exemplary embodiment depicted in FIG. 5, the actuator can also be in direct contact with a vibratory mechanism such that the actuator vibrates in response to activation of the vibratory mechanism.

In the embodiment depicted in FIGS. 1-4C, the actuator is a cable 50 that extends from the handle 20 to the end effector 40 within (or in other embodiments around) the elongate shaft 30. The cable 50 can be received at the distal portion of the handle 20 and it extends proximally through the collar 28 of the shuttle assembly 25. The cable 50 can be securely fastened to the collar 28 such that the actuator 50 cannot translate axially with respect to the collar 28. The cable 50 can also extend further proximally from the collar 28, for example, to the rotation knob 26 such that the proximal most end of the actuator is coupled to or disposed within the rotation knob 26. As discussed above, rotation of the knob 26 can be effective to rotate the cable 50.

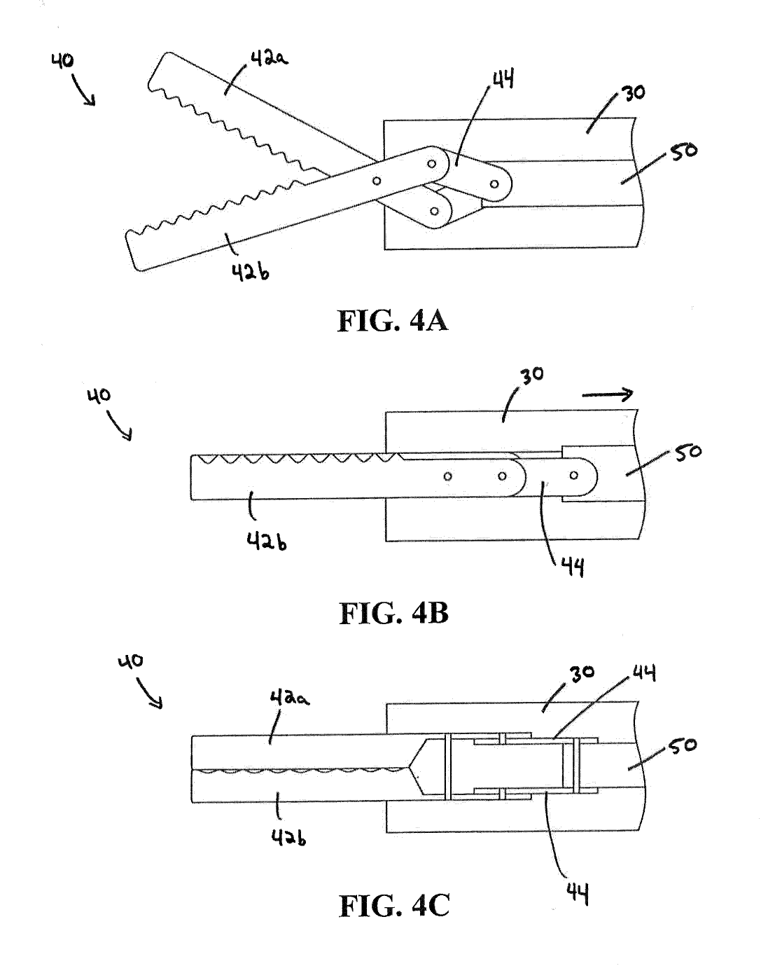

The distal portion of the cable 50 can extend through the shaft 30 such that the distal end of the cable 50 can couple to an end effector 40 disposed at a distal end of the shaft 30. As discussed elsewhere herein, longitudinal (i.e., axial) movement of the cable 50 relative to the shaft 30 can be effective to actuate the end effector 40, for example, as shown in FIGS. 4A and 4B. Alternatively or in addition, rotational movement of the cable 50 relative to the shaft 30 can be effective to rotate the end effector 40, for example, as shown in FIGS. 4B and 4C.

The end effector coupled to or disposed on the distal end of the elongate shaft can also have a variety of configurations for performing various procedures, such as fastening, manipulating, cutting, treating, or otherwise effecting tissue. A person skilled in the art will appreciate that, while the methods and devices are described in connection with surgical scissors, the concepts can be applied to a variety of other surgical, therapeutic, or diagnostic end effectors known in the art anf modified in light of the teachings herein.

In the depicted embodiment, the end effector of the device 10 is in the form of a pair of surgical scissors 40. The scissors 40 generally include opposed jaws 42a, 42b that are movable between an open configuration as shown in FIG. 4A and a closed configuration as shown in FIG. 4B. The opposed jaws 42a, 42b are configured to engage tissue therebetween such that actuation from the open configuration to the closed configuration is effective to cut tissue.

The opposed jaws 42a, 42b can be coupled to the distal end of the actuator 50 through linkage members 44 such that longitudinal (i.e., axial) movement of the actuator 50 through the shaft 30 is effective to move the opposed jaws 42a, 42b between the open and closed configurations. By way of example, proximal axial movement of the actuator 50 can pull the linkage members 44 proximally to close the opposed jaws 42a, 42b. On the other hand, distal movement of the actuator 50 can be effective to open the opposed jaws 42a, 42b. As will be appreciated by a person skilled in the art, in some embodiments, the opposed jaws can be selectively biased to one of an open configuration or a closed configuration.

Rotation of the actuator 50 by way of the rotatable knob 26 in a first direction (e.g., clockwise), for example, can be effective to rotate the end effector from the position shown in FIG. 4B to that of FIG. 4C. Rotation of the actuator 50 in an opposite direction (e.g., counterclockwise) can be effective to rotate the end effector from the position shown in FIG. 4B to that of FIG. 4C.

The surgical devices described herein also include a vibratory mechanism coupled thereto for vibrating the shaft and/or the actuator to reduce the frictional forces generated between the shaft and the actuator, particularly when the shaft is inserted into a patient, thereby reducing the force required to actuate the end effector. A person skilled in the art will appreciate that the vibratory mechanism can have a variety of configurations, but generally is coupled to the surgical instrument such that the vibrations produced by the vibratory mechanism are effective to vibrate at least one of the shaft and the actuator. Similarly, a vibratory mechanism could be utilized to reduce frictional effects in handle components as well. By way of non-limiting example, the vibratory mechanism can be a mechanical, electromechanical, electromagnetic, or ultrasonic vibrator to effect vibrations in the shaft and/or the actuator. The vibratory mechanism can also include various other components necessary for operation, such as a power source (e.g., battery), control circuitry, and a switch (e.g., push-button, slide switches, rotatable knobs, or otherwise).

As will be understood by one skilled in the art, the vibratory mechanism can be coupled to any location on or within the surgical device 10 that is effective to directly or indirectly vibrate the shaft and/or actuator. By way of non-limiting example, the vibratory mechanism can be disposed within or on the handle, on or within the shaft, or on or within the actuator. The vibratory mechanism can be coupled to the surgical device in a variety of ways. For example, the vibratory mechanism can be integrally formed with the surgical device, manually held against the device, or fixedly or replaceably coupled to the surgical device. By way of non-limiting example, the vibratory mechanism can be welded, molded, or glued to the surgical device. Alternatively, or in addition, screws, bolts, clips, tape, or other fastening techniques known in the art can also be employed to couple the vibratory mechanism to the surgical device.

In the embodiment shown of FIGS. 1-4C, the vibratory mechanism 60 includes a motor 62 and a piston 64. With specific reference to FIG. 3, the motor 62 is operatively connected to a power source (not shown) such that activation of the motor 62 is effective to oscillate the piston 64. The piston 64 can directly contact the proximal end of the shaft 30 on each stroke, thereby effecting vibrations down the length of the shaft 30. Although the piston 64 is shown contacting the proximal end of the shaft 30, the piston 64 and shaft 30 need not be in direct physical contact in order to vibrate the shaft 30. For example, a buffer capable of transmitting vibrations can be placed between the piston 64 and the shaft 30 to protect the shaft 30 from damage caused by repeated contact with the piston 64. In some embodiments, the vibratory mechanism 60 can be surrounded by or proximate to a dampener in order to reduce the vibrations transmitted to portions of the device 10 other than the shaft 30. Alternatively or in addition, dampeners may be placed distant from the vibratory mechanism such as, for example, a dampener 27 disposed within the grip 22 such that the operator's grasp is isolated from the vibrations.

The vibratory mechanism 60 can be selectively activated in any number of ways, including by one or more switches, foot pedals, remote controls, computers, sensors, or any other activation mechanism known in the art. For example, the motor can be coupled via electrical wires 68 to a switch (not shown) that can be disposed on an external surface of the handle 20. An operator can activate (i.e., turn on) the vibratory mechanism 60 by toggling the switch. In addition to manual activation, the vibratory mechanism 60 can be automatically actuated, for example, by initiation of actuation of the actuator 50. For example, in the embodiment depicted in FIG. 3, the motor 62 can be configured to activate when the trigger 24 is squeezed or the rotatable knob 26 is rotated. By way of non-limiting example, an electrical contact (not shown) disposed on the arms 24a, 24b of the trigger 24 can complete an electrical circuit such that the vibratory mechanism 60 is activated when the arms 24a, 24b (and the electrical contact disposed thereon) are moved with the actuation of the trigger 24. Similarly, as will be appreciated by a person skilled in the art, rotation of the knob 26 can be effective to signal the vibratory mechanism to activate such that the vibratory mechanism is activated concurrently with actuation of the actuator 50.

Referring now to FIG. 5, another embodiment of a surgical device 510 is provided. The surgical device 510 is similar to device 10 except that the vibratory mechanism is configured to directly contact and vibrate the actuator 550. In this embodiment, a motor 562 is disposed in the handle 520 such that activation of the motor 562 is effective to oscillate the piston 564. The piston 564 can contact the proximal end of the actuator 550 on each stroke, thereby effecting vibrations down the length of the actuator 550. In some embodiments, the surgical device 510 can include a dampener (not shown) to reduce the vibrations transmitted to portions of the device 510 other than the shaft 530 such that the operator's grasp is isolated from the vibrations.

As will be appreciated by a person skilled in the art, any electromechanical motor effective to vibrate the shaft and/or actuator can be used. For example, though the motor 562 depicted in FIG. 5 includes a piston 564 that intermittently contacts the actuator 550, the piston 564 could alternatively be coupled to the actuator 550 such that the actuator 550 remains coupled to the piston 564 during the entire reciprocating movement of the piston 564.

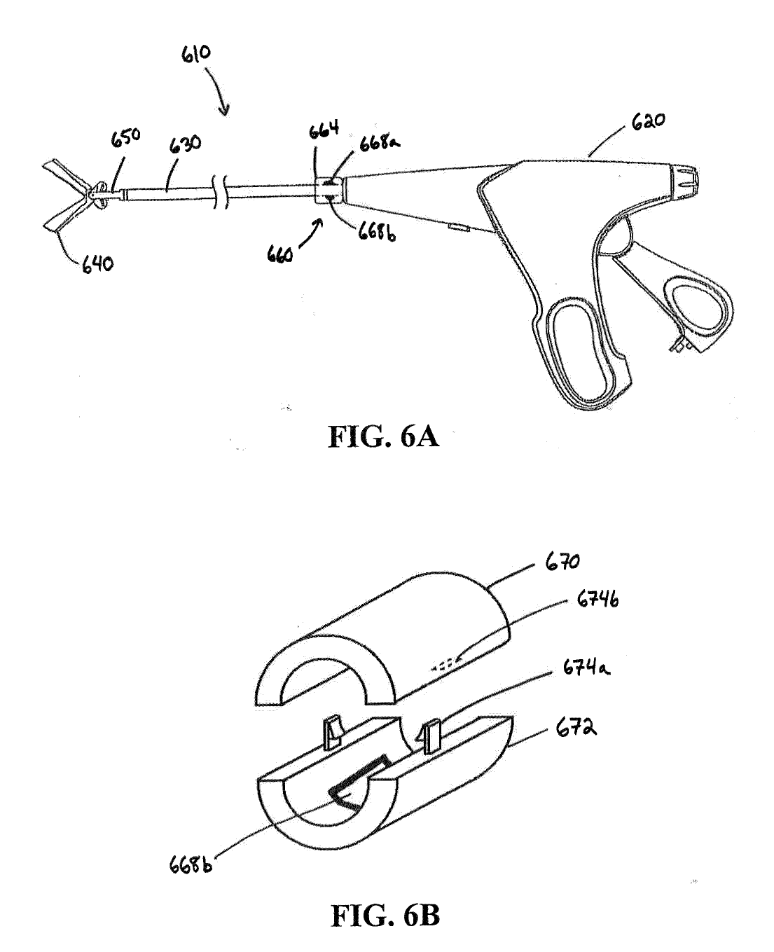

Alternatively, or in addition to the electromechanical vibratory mechanisms described above, an ultrasonic transducer can be used to vibrate the shaft and/or actuator. Referring now to FIGS. 6A and 6B, another embodiment of a surgical device 610 is depicted. The surgical device 610 is substantially similar to surgical device 10 except that the vibratory mechanism 660 is located external to the handle 620. The vibratory mechanism 660 includes a sleeve 664 that surrounds the periphery of the shaft 630. Disposed within the sleeve 664 is one or more ultrasonic transducers 668a, 668b (and optionally, the transducer's control circuitry) such that activation of the ultrasonic transducers 668a, 668b is effective to transmit ultrasonic vibrations down the length of the shaft 630.

The ultrasonic transducers 668a, 668b can have a variety of configurations, but in one embodiment, the control circuitry can be configured to selectively produce an alternating voltage across one or more piezoelectric crystals in the ultrasonic transducers 668a, 668b, thereby causing the crystals to resonate and produce high frequency sound waves. In certain embodiments, the frequency of the sound waves is in the ultrasonic range. The emission of high frequency sound waves from the ultrasonic transducer 668a, 668b can cause the shaft 630, or at least portions thereof, to vibrate. The ultrasonic transducers can be electrically wired to an activator as described above in reference to FIG. 3, or alternatively, can be activated remotely, for example, by remote control.

As discussed above, the vibratory mechanism 660 can be coupled to the surgical device 610 at any location effective to vibrate at least one of the shaft 630 and the actuator 630. Additionally, the vibratory mechanism 660 can be integrally formed, or permanently or removably coupled to the surgical device. By way of example, the vibratory mechanism 660 can snap on or slide onto the shaft 630. For example, the vibratory mechanism can be provided as a snap-on or slide-on device for attachment to a shaft of any surgical device where vibration is desired. However, in the embodiment shown in FIG. 6A, the sleeve 664 includes an upper segment 670 and a lower segment 672 that are removably and replaceably coupled to the shaft 630. Each segment 670, 672 includes a concave recess configured to seat the shaft 630 therein, and a coupling mechanism to allow the segments to couple together. As shown in detail in FIG. 6B, the lower segment 672 includes a flange 674a that can be securely received within a recess 674b (shown in phantom) formed in the upper segment 670. One skilled in the art will appreciate that any number of engagement mechanisms (e.g., snap-fit couplings, threading, interference fit, adhesive etc.) can be used to removably couple the segments 670, 672 to one another.

In an alternative embodiment, the sleeve 664 can, instead of or in addition to the configuration described directly above, line the internal wall of the shaft 630 such that the vibratory mechanism 660 is disposed within the elongate shaft. In this manner, the vibratory mechanism can be configured to directly contact the shaft 630, the actuator 650 disposed therethrough, or both to thereby transmit vibrations.

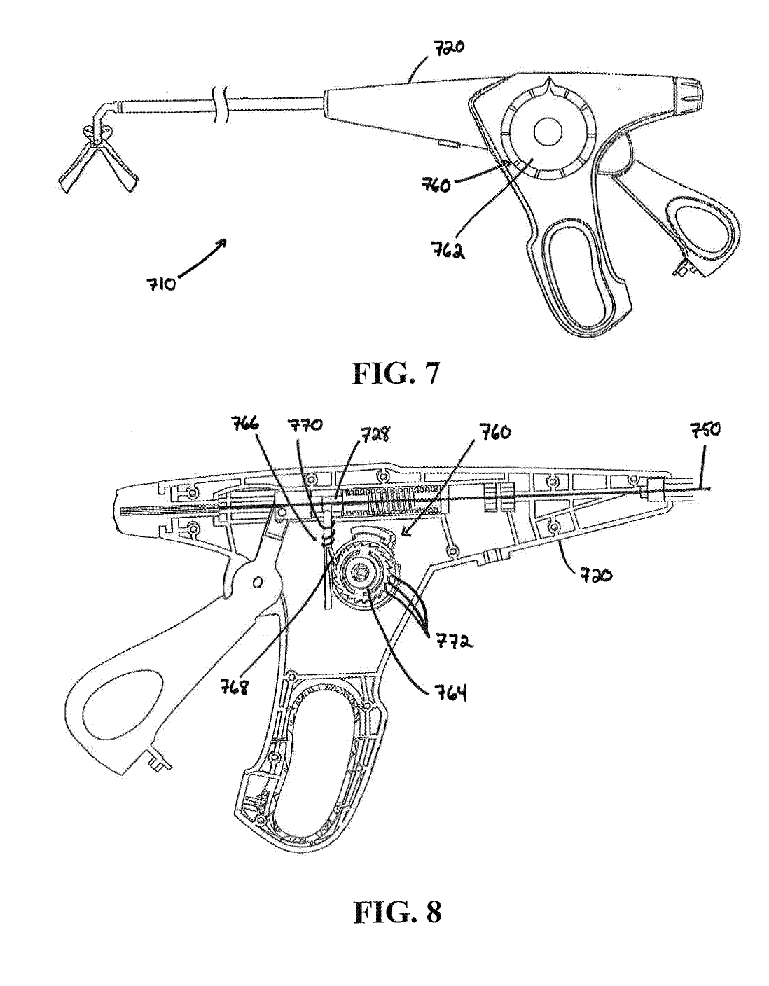

Referring now to FIGS. 7 and 8, another embodiment of a surgical device 710 is depicted. The surgical device 710 is substantially similar to surgical device 10 except that the vibratory mechanism is a mechanical vibrator 760 which can be manually operated. FIG. 7 also illustrate an end effector that can articulate relative to the shaft. The mechanical vibrator 760 includes an external rotatable dial 762, an internal gear 764, and a slide mechanism 766 configured to contact (directly or indirectly) the actuator 750 to cause vibrations therein.

The external dial 762 is rotatably coupled to the handle 720 such that rotation of the dial 762 by the operator is effective to turn the internal gear 764. Also internal to the handle 720, is a slide mechanism 766 having a detent 768 disposed thereon. A spring 770 disposed around the slide mechanism 766 and extending between the detent 768 and the actuator 750 (through the collar 728 surrounding the actuator 750) biases the slide mechanism 766 out of contact with the collar 728. Rotation of the external dial 762 causes corresponding rotation of the internal gear 764 such that the gear teeth 772 contact the detent 768, thereby sliding the slide mechanism 766 into contact with the collar 728. Continual rotation of the external dial 762 results in oscillation of the detent 768 and movement of the slide mechanism 766 into and out of contact with the collar 728, thereby transmitting vibrations to the actuator 750. While a rotatable dial is shown, in other embodiments the mechanical vibratory mechanism can be operatively associated with the trigger such that movement of the trigger cause corresponding movement of the vibratory mechanism.

In use, the various devices disclosed herein can be delivered to an internal surgical site through a natural orifice or a surgical incision. By way of non-limiting example, FIG. 1 depicts the end effector 40 on the distal end of the flexible elongate shaft 30 being delivered to the stomach 8 by advancing the flexible elongate shaft 30 through a tortuous body lumen. Once positioned at or adjacent the surgical site, the flexible elongate actuator 50 that extends through the shaft 30 can be actuated (i.e., moved longitudinally and/or rotationally relative to the shaft 30) to thereby actuate the end effector 40. Actuation of the end effector can be effective, for example, to properly position the end effector 40 and/or perform the surgical manipulation of interest (e.g., open and close opposed jaws 42a, 42b of the end effector 40 to cut tissue). By way of non-limiting example, the actuator 50 can be rotated to rotate the end effector 40. Alternatively, or in addition to, the actuator can be translated longitudinally (i.e., axially) thorough the shaft when the actuator 50 is actuated.

According to the teachings herein, a vibratory mechanism coupled to the surgical device 10 can be activated to vibrate at least one of the shaft 30 and the actuator 50. The vibratory mechanism 60 can be activated prior to and/or concurrently with actuating the flexible elongate actuator 50. Vibration of the shaft 30 and/or the actuator 50 extending therethrough can be effective to reduce a frictional force generated between the shaft and the actuator, particularly when the shaft is positioned within the tortuous body lumen, thereby reducing a force necessary to actuate the end effector 40. For example, activating the vibratory mechanism can convert the static frictional force to a dynamic frictional force. By the same token, the surgical device 10 can include electrical contacts to automatically activate the vibratory mechanism 60 whenever the trigger 24 is actuated.

The devices disclosed herein can also be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning and/or replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. It is preferred that the device is sterilized. This can be done by any number of ways known to those skilled in the art including beta or gamma radiation, ethylene oxide, steam, e-beam, etc.

One skilled in the art will appreciate further features and advantages of the invention based on the above-described embodiments. Accordingly, the invention is not to be limited by what has been particularly shown and described, except as indicated by the appended claims.

What is claimed is:

1. A surgical instrument, comprising:
a flexible elongate shaft having a proximal end coupled to a handle and a distal end configured to be inserted into a patient;
an end effector coupled to the distal end of the shaft and configured to effect tissue;
a flexible elongate actuator extending between the handle and the end effector, the actuator being configured to actuate the end effector; and
a vibratory mechanism coupled to the surgical instrument and configured to vibrate at least one of the shaft and the actuator to reduce a frictional force generated between the shaft and the actuator when the shaft is inserted through a tortuous body lumen, thereby reducing a force necessary to actuate the end effector.

2. The apparatus of claim 1, wherein the vibratory mechanism is disposed within the elongate shaft.

3. The apparatus of claim 1, wherein the vibratory mechanism comprises an ultrasonic transducer.

4. The apparatus of claim 1, wherein the vibratory mechanism comprises a mechanical vibrator.

5. The apparatus of claim 1, wherein the handle includes a dampener to reduce vibration transmitted to the handle.

6. The apparatus of claim 1, wherein the actuator comprises a flexible elongate cable.

7. The apparatus of claim 1, wherein the actuator is rotatable relative to the shaft such that the actuator is configured to apply a rotational force to the end effector to rotate the end effector.

8. The apparatus of claim 1, wherein the actuator is configured to translate longitudinally relative to the shaft.

9. The apparatus of claim 1, wherein actuation comprises at least one of rotation of the end effector relative to the elongate shaft, articulation of the end effector relative to the elongate shaft, and opening and closing of opposed jaws of the end effector.

10. The apparatus of claim 1, wherein the end effector comprises opposed jaws and the actuator is configured to move the opposed jaws between open and closed positions.

11. The apparatus of claim 1, wherein the vibratory mechanism is removably and replaceably coupled to the shaft.

12. An surgical device, comprising:
a handle;
a flexible elongate shaft extending distally from the handle;
an end effector disposed on a distal end of the flexible elongate shaft and configured to effect tissue;
a flexible actuator extending between the handle and the end effector, the flexible actuator being movable relative to the elongate shaft to effect movement of the end effector; and
a vibratory mechanism associated with at least one of the shaft and the actuator and configured to convert a static frictional force, generated between the shaft and the actuator when the shaft is disposed through a tortuous body lumen, to a dynamic frictional force to thereby reduce a force necessary to actuate the end effector.

13. The apparatus of claim 12, wherein the vibratory mechanism is disposed within the elongate shaft.

14. The apparatus of claim 12, wherein the vibratory mechanism is removably coupled around a perimeter of the elongate shaft.

15. The apparatus of claim 12, wherein the vibratory mechanism comprises one of a motor, an ultrasonic transducer, and a mechanical vibrator.

16. A surgical method, comprising:
advancing a flexible elongate shaft of a surgical instrument through a tortuous body lumen to position an end effector on a distal end of the elongate shaft within the body; and
actuating a flexible elongate actuator extending between a handle coupled to a proximal end of the elongate shaft and the end effector to actuate the end effector;
wherein a vibratory mechanism coupled to the surgical instrument vibrates at least one of the shaft and the actuator to reduce a frictional force generated between the shaft and the actuator when the shaft is positioned within the tortuous body lumen, thereby reducing a force necessary to actuate the end effector.

17. The method of claim 16, wherein the flexible elongate actuator translates longitudinally through the elongate shaft when the actuator is actuated.

18. The method of claim 16, wherein actuating the flexible elongate actuator opens and closes opposed jaws of the end effector.

19. The method of claim 16, wherein the flexible elongate actuator rotates during actuation to rotate the end effector.

20. The method of claim 16, further comprises activating the vibratory mechanism prior to actuating the flexible elongate actuator.

21. The method of claim 16, wherein the vibratory mechanism is activated concurrently with actuation of the actuator.
